(19) United States
(12) Patent Application Publication
(10) Pub. No.: US 2023/0390056 A1
(43) Pub. Date: Dec. 7, 2023

(54) Balloon Expandable Valve Securement Aids

(71) Applicant: St. Jude Medical, Cardiology Division, Inc., St. Paul, MN (US)

(72) Inventors: Tracee Eidenschink, Wayzata, MN (US); Michael Shane Morrissey, St. Paul, MN (US); Peter J. Ness, Minneapolis, MN (US); Daniel J. Klima, Andover, MN (US); Tyler Govek, Minneapolis, MN (US); Kristopher Henry Vietmeier, Monticello, MN (US)

(73) Assignee: St. Jude Medical, Cardiology Division, Inc., St. Paul, MN (US)

(21) Appl. No.: 18/327,469

(22) Filed: Jun. 1, 2023

Related U.S. Application Data
(60) Provisional application No. 63/349,244, filed on Jun. 6, 2022.

(51) Int. Cl. A61F 2/24

(52) U.S. Cl. CPC A61F 2/2418; A61F 2/2436; A61F 2220/0075; A61F 2230/0002

(57) ABSTRACT

In some embodiments, a prosthetic heart valve system, includes a prosthetic heart valve including a stent, a cuff and a plurality of leaflets, the cuff and the plurality of leaflets forming a valve assembly, and the stent having one or more retention tabs, and a delivery device having an inner shaft and an expandable balloon transitionable between a deflated state and an inflated state, the delivery device having a hub with one or more receivers to accept the one or more retention tabs.

This application claims priority to U.S. Provisional Patent Application No. 63/349,244, filed Jun. 6, 2022, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves, including surgical heart valves and collapsible/expandable heart valves intended for transcatheter aortic valve replacement (“TAVR”) or transcatheter mitral valve replacement (“TMVR”), are well known in the patent literature. Surgical or mechanical heart valves may be sutured into a native annulus of a patient during an open-heart surgical procedure, for example. Collapsible/expandable heart valves may be delivered into a patient via a tube-like delivery apparatus such as a catheter, a trocar, a laparoscopic instrument, or the like to avoid a more invasive procedure such as full open-chest, open-heart surgery. As used herein, reference to a “collapsible/expandable” heart valve includes heart valves that are formed with a small cross-section that enables them to be delivered into a patient through a tube-like delivery apparatus in a minimally invasive procedure, and then expanded to an operable state once in place, as well as heart valves that, after construction, are first collapsed to a small cross-section for delivery into a patient and then expanded to an operable size once in place in the valve annulus.

Collapsible/expandable prosthetic heart valves typically take the form of a one-way valve structure (often referred to herein as a valve assembly) mounted to/within an expandable stent. In general, these collapsible/expandable heart valves include a self-expanding or balloon-expandable stent, often made of nitinol or another shape-memory metal or metal alloy (for self-expanding stents) or steel or cobalt chromium (for balloon-expandable stents). Existing collapsible/expandable TAVR devices have been known to use different configurations of stent layouts—including straight vertical struts connected by “V”s as illustrated in U.S. Pat. No. 8,454,685, or diamond-shaped cell layouts as illustrated in U.S. Pat. No. 9,326,856, both of which are hereby incorporated herein by reference. The one-way valve assembly mounted to/within the stent includes one or more leaflets, and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

When self-expandable prosthetic heart valves are delivered into a patient to replace a malfunctioning native heart valve, the self-expandable prosthetic heart valve is almost always maintained in the collapsed condition within a capsule of the delivery device. While the capsule may ensure that the prosthetic heart valve does not self-expand prematurely, the overlying capsule (with or without the help of additional internal retaining features) helps ensure that the prosthetic heart valve does not come into contact with any tissue prematurely, as well as helping to make sure that the prosthetic heart valve stays in the desired position and orientation relative to the delivery device during delivery. However, balloon expandable prosthetic heart valves are typically crimped onto the balloon of a delivery device without a separate capsule that overlies and/or protects the prosthetic heart valve. One reason for this is that space is always at a premium in transcatheter prosthetic heart valve delivery devices and systems, and adding a capsule in addition to the prosthetic valve and the underlying balloon may not be feasible given the size profile requirements of these procedures.

During crimping, the prosthetic heart valve may move relative to the crimping device. This movement may be translation, rotational or a combination of the two. A similar concern is present when loading a prosthetic heart valve onto a balloon of a delivery device, and during delivery of the prosthetic heart valve, especially as the delivery device navigates the tortuous path to its implant location.

### BRIEF SUMMARY OF THE DISCLOSURE

In some embodiments, a prosthetic heart valve system, includes a prosthetic heart valve including a stent, a cuff and a plurality of leaflets, the cuff and the plurality of leaflets forming a valve assembly, and the stent having one or more retention tabs, and a delivery device having an inner shaft and an expandable balloon transitionable between a deflated state and an inflated state, the delivery device having a hub with one or more receivers to accept the one or more retention tabs.

In some embodiments, a prosthetic heart valve system, includes a prosthetic heart valve including a stent, a cuff and a plurality of leaflets, the cuff and the plurality of leaflets forming a valve assembly; and a delivery device having an inner shaft and an expandable balloon transitionable between a deflated state and an inflated state, the delivery device having a garter system including at least one anchor capable of mating with a cell of the stent, and at least one coupler connected to the at least one anchor.

### BRIEF DESCRIPTION OF THE DRAWINGS

FIG. 1A is a perspective view of a stent of a prosthetic heart valve according to an embodiment of the disclosure.

FIG. 1B is a schematic front view of a section of the stent of FIG. 1A.

FIG. 1C is a schematic front view of a section of a stent according to an alternate embodiment of the prosthetic heart valve of FIG. 1A.

FIGS. 1D-E are front views of the stent section of FIG. 1C in a collapsed and expanded state, respectively.

FIGS. 1F-G are side views of a portion of the stent according to the embodiment of FIG. 1C in a collapsed and expanded state, respectively.

FIG. 1H is a flattened view of the stent according to the embodiment of FIG. 1C, as if cut and rolled flat.

FIGS. 1I-J are front and side views, respectively, of a prosthetic heart valve including the stent of FIG. 1C.

FIG. 1K illustrates the view of FIG. 1H with an additional outer cuff provided on the stent.

FIGS. 2A-B illustrates a prosthetic heart valve PHV, crimped over a balloon in the deflated and inflated conditions.

FIGS. 3A-C are schematic perspective and side views of a prosthetic heart valve having retention tabs, and the same prosthetic heart valve being loaded into a delivery device and a crimping tool.

FIGS. 4A-B are schematic top views of another example of a prosthetic heart valve in use with a valve cradle.

FIGS. 5A-B are schematic representations of a prosthetic heart valve in use with a garter system, in expanded and collapsed condition, respectively.

FIGS. 6A-B are schematic side views of prosthetic heart valves retained with a garter system onto a shaft of a delivery device.

FIG. 7 is a schematic representation of one example of an open cell for use with the garter system.

### DETAILED DESCRIPTION

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing the valve disclosed herein, however, it should be noted that the use of the valve is not limited to the intended position and orientation, but may be deployed in any type of lumen or passageway. For example, although the prosthetic heart valve is described herein as a prosthetic aortic valve, the same or similar structures and features can be employed in other heart valves, such as the pulmonary valve, the mitral valve, or the tricuspid valve. Further, the term “proximal,” when used in connection with a delivery device or system, refers to a direction relatively close to the user of that device or system when being used as intended, while the term “distal” refers to a direction relatively far from the user of the device. In other words, the leading end of a delivery device or system is positioned distal to a trailing end of the delivery device or system, when being used as intended. As used herein, the terms “substantially,” “generally,” “approximately,” and “about” are intended to mean that slight deviations from absolute are included within the scope of the term so modified. As used herein, the stent may assume an “expanded state” and a “collapsed state,” which refer to the relative radial size of the stent.

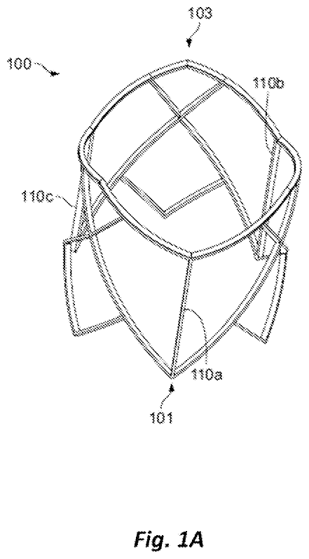

FIG. 1A illustrates a perspective view of a stent 100 of a prosthetic heart valve according to an embodiment of the disclosure. Stent 100 may include a frame extending in an axial direction between an inflow end 101 and an outflow end 103. Stent 100 includes three generally symmetric sections, wherein each section spans about 120 degrees around the circumference of stent 100. Stent 100 includes three vertical struts 110a, 110b, 110c, that extend in an axial direction substantially parallel to the direction of blood flow through the stent, which may also be referred to as a central longitudinal axis. Each vertical strut 110a, 110b, 110c may extend substantially the entire axial length between the inflow end 101 and the outflow end 103 of the stent 100, and may be disposed between and shared by two sections. In other words, each section is defined by the portion of stent 100 between two vertical struts. Thus, each vertical strut 110a, 110b, 110c is also separated by about 120 degrees around the circumference of stent 100. It should be understood that, if stent 100 is used in a prosthetic heart valve having three leaflets, the stent may include three sections as illustrated. However, in other embodiments, if the prosthetic heart valve has two leaflets, the stent may only include two of the sections.

FIG. 1B illustrates a schematic view of a stent section 107 of stent 100, which will be described herein in greater detail and which is representative of all three sections. Stent section 107 depicted in FIG. 1B includes a first vertical strut 110a and a second vertical strut 110b. First vertical strut 110a extends axially between a first inflow node 102a and a first outer node 135a. Second vertical strut 110b extends axially between a second inflow node 102b and a second outer node 135b. As is illustrated, the vertical struts 110a, 110b may extend almost the entire axial length of stent 100. In some embodiments, stent 100 may be formed as an integral unit, for example by laser cutting the stent from a tube. The term “node” may refer to where two or more struts of the stent 100 meet one another. A pair of sequential inverted V's extends between inflow nodes 102a, 102b, which includes a first inflow inverted V 120a and a second inflow inverted V 120b coupled to each other at an inflow node 105. First inflow inverted V 120a comprises a first outer lower strut 122a extending between first inflow node 102a and a first central node 125a. First inflow inverted V 120a further comprises a first inner lower strut 124a extending between first central node 125a and inflow node 105. A second inflow inverted V 120b comprises a second inner lower strut 124b extending between inflow node 105 and a second central node 125b. Second inflow inverted V 120b further comprises a second outer lower strut 122b extending between second central node 125b and second inflow node 102b. Although described as inverted V's, these structures may also be described as half-cells, each half cell being a half-diamond cell with the open portion of the half-cell at the inflow end 101 of the stent 100.

Stent section 107 further includes a first central strut 130a extending between first central node 125a and an upper node 145. Stent section 107 also includes a second central strut 130b extending between second central node 125b and upper node 145. First central strut 130a, second central strut 130b, first inner lower strut 124a and second inner lower strut 124b form a diamond cell 128. Stent section 107 includes a first outer upper strut 140a extending between first outer node 135 and a first outflow node 104a. Stent section 107 further includes a second outer upper strut 140b extending between second outer node 135b and a second outflow node 104b. Stent section 107 includes a first inner upper strut 142a extending between first outflow node 104a and upper node 145. Stent section 107 further includes a second inner upper strut 142b extending between upper node 145 and second outflow node 104b. Stent section 107 includes an outflow inverted V 114 which extends between first and second outflow nodes 104a, 104b. First vertical strut 110a, first outer upper strut 140a, first inner upper strut 142a, first central strut 130a and first outer lower strut 122a form a first generally kite-shaped cell 133a. Second vertical strut 110b, second outer upper strut 140b, second inner upper strut 142b, second central strut 130b and second outer lower strut 122b form a second generally kite-shaped cell 133b. First and second kite-shaped cells 133a, 133b are symmetric and opposite each other on stent section 107. Although the term “kite-shaped,” is used above, it should be understood that such a shape is not limited to the exact geometric definition of kite-shaped. Outflow inverted V 114, first inner upper strut 142a and second inner upper strut 142b form upper cell 134. Upper cell 134 is generally kite-shaped and axially aligned with diamond cell 128 on stent section 107. It should be understood that, although designated as separate struts, the various struts described herein may be part of a single unitary structure as noted above. However, in other embodiments, stent 100 need not be formed as an integral structure and thus the struts may be different structures (or parts of different structures) that are coupled together.

FIG. 1C illustrates a schematic view of a stent section 207 according to an alternate embodiment of the disclosure. Unless otherwise stated, like reference numerals refer to like elements of above-described stent 100 but within the 200-series of numbers. Stent section 207 is substantially similar to stent section 107, including inflow nodes 202a, 202b, vertical struts 210a, 210b, first and second inflow inverted V's 220a, 220b and outflow nodes 204a, 204b. The structure of stent section 207 departs from that of stent section 107 in that it does not include an outflow inverted V. The purpose of an embodiment having such structure of stent section 207 shown in FIG. 1C is to reduce the required force to expand the outflow end 203 of the stent 200, compared to stent 100, to promote uniform expansion relative to the inflow end 201. Outflow nodes 204a, 204b are connected by a properly oriented V formed by first inner upper strut 242a, upper node 245 and second inner upper strut 242b. In other words, struts 242a, 242b may form a half diamond cell 234, with the open end of the half-cell oriented toward the outflow end 203. Half diamond cell 234 is axially aligned with diamond cell 228. Adding an outflow inverted V coupled between outflow nodes 204a, 204b contributes additional material that increases resistance to modifying the stent shape and requires additional force to expand the stent. The exclusion of material from outflow end 203 decreases resistance to expansion on outflow end 203, which may promote uniform expansion of inflow end 201 and outflow end 203. In other words, the inflow end 201 of stent 200 does not include continuous circumferential structure, but rather has mostly or entirely open half-cells with the open portion of the half-cells oriented toward the inflow end 201, whereas most of the outflow end 203 includes substantially continuous circumferential structure, via struts that correspond with struts 140a, 140b. All else being equal, a substantially continuous circumferential structure may require more force to expand compared to a similar but open structure. Thus, the inflow end 101 of stent 100 may require more force to radially expand compared to the outflow end 103. By omitting inverted V 114, resulting in stent 200, the force required to expand the outflow end 203 of stent 200 may be reduced to an amount closer to the inflow end 201.

FIG. 1D shows a front view of stent section 207 in a collapsed state and FIG. 1E shows a front view of stent section 207 in an expanded state. It should be understood that stent 200 in FIGS. 1D-E is illustrated with an opaque tube extending through the interior of the stent, purely for the purpose of helping illustrate the stent, and which may represent a balloon over which the stent section 207 is crimped. As described above, a stent comprises three symmetric sections, each section spanning about 120 degrees around the circumference of the stent. Stent section 207 illustrated in FIGS. 1D-E is defined by the region between vertical struts 210a, 210b. Stent section 207 is representative of all three sections of the stent. Stent section 207 has an arcuate structure such that when three sections are connected, they form one complete cylindrical shape. FIGS. 1F-G illustrate a portion of the stent from a side view. In other words, the view of stent 200 in FIGS. 1F-G is rotated about 60 degrees compared to the view of FIGS. 1D-E. The view of the stent depicted in FIGS. 1F-G is centered on vertical strut 210b showing approximately half of each of two adjacent stent sections 207a, 207b on each side of vertical strut 210b. Sections 207a, 207b surrounding vertical strut 210b are mirror images of each other. FIG. 1F shows stent sections 207a, 207b in a collapsed state whereas FIG. 1G shows stent sections 207a, 207b in an expanded state.

FIG. 1H illustrates a flattened view of stent 200 including three stent sections 207a, 207b, 207c, as if the stent has been cut longitudinally and laid flat on a table. As depicted, sections 207a, 207b, 207c are symmetric to each other and adjacent sections share a common vertical strut. As described above, stent 200 is shown in a flattened view, but each section 207a, 207b, 207c has an arcuate shape spanning 120 degrees to form a full cylinder. Further depicted in FIG. 1H are leaflets 250a, 250b, 250c coupled to stent 200. However, it should be understood that only the connection of leaflets 250a-c is illustrated in FIG. 1H. In other words, each leaflet 250a-c would typically include a free edge, with the free edges acting to coapt with one another to prevent retrograde flow of blood through the stent 200, and the free edges moving radially outward toward the interior surface of the stent to allow antegrade flow of blood through the stent. Those free edges are not illustrated in FIG. 1H. Rather, the attached edges of the leaflets 250a-c are illustrated in dashed lines in FIG. 1H. Although the attachment may be via any suitable modality, the attached edges may be preferably sutured to the stent 200 and/or to an intervening cuff or skirt between the stent and the leaflets 250a-c. Each of the three leaflets 250a, 250b, 250c, extends about 120 degrees around stent 200 from end to end and each leaflet includes a belly that may extend toward the radial center of stent 200 when the leaflets are coapted together. Each leaflet extends between the upper nodes of adjacent sections. First leaflet 250a extends from first upper node 245a of first stent section 207a to second upper node 245b of second stent section 207b. Second leaflet 250b extends from second upper node 245b to third upper node 245c of third stent section 207c. Third leaflet 250c extends from third upper node 245c to first upper node 245a. As such, each upper node includes a first end of a first leaflet and a second end of a second leaflet coupled thereto. In the illustrated embodiment, each end of each leaflet is coupled to its respective node by suture. However, any coupling means may be used to attach the leaflets to the stent. It is further contemplated that the stent may include any number of sections and/or leaflets. For example, the stent may include two sections, wherein each section extends 180 degrees around the circumference of the stent. Further, the stent may include two leaflets to mimic a bicuspid valve. Further, it should be noted that each leaflet may include tabs or other structures (not illustrated) at the junction between the free edges and attached edges of the leaflets, and each tab of each leaflet may be coupled to a tab of an adjacent leaflet to form commissures. In the illustrated embodiment, the leaflet commissures are illustrated attached to nodes where struts intersect. However, in other embodiments, the stent 200 may include commissure attachment features built into the stent to facilitate such attachment. For example, commissures attachment features may be formed into the stent 200 at nodes 245a-c, with the commissure attachment features including one or more apertures to facilitate suturing the leaflet commissures to the stent. Further, leaflets 250a-c may be formed of a biological material, such as animal pericardium, or may otherwise be formed of synthetic materials, such as ultra-high molecular weight polyethylene (UHMWPE).

FIGS. 1I-J illustrate prosthetic heart valve 206, which includes stent 200, a cuff 260 coupled to stent 200 (for example via sutures) and leaflets 250a, 250b, 250c attached to stent 200 and/or cuff 260 (for example via sutures). Prosthetic heart valve 206 is intended for use in replacing an aortic valve, although the same or similar structures may be used in a prosthetic valve for replacing other heart valves. Cuff 260 is disposed on a luminal or interior surface of stent 200, although the cuff could be disposed alternately or additionally on an abluminal or exterior surface of the stent. The cuff 260 may include an inflow end disposed substantially along inflow end 201 of stent 200. FIG. 1I shows a front view of valve 206 showing one stent portion 207 between vertical struts 210a, 210b including cuff 260 and an outline of two leaflets 250a, 250b sutured to cuff 260. Different methods of suturing leaflets to the cuff as well as the leaflets and/or cuff to the stent may be used, many of which are described in U.S. Pat. No. 9,326,856 which is hereby incorporated by reference. In the illustrated embodiment, the upper (or outflow) edge of cuff 260 is sutured to first central node 225a, upper node 245 and second central node 225b, extending along first central strut 230a and second central strut 230b. The upper (or outflow) edge of cuff 260 continues extending approximately between the second central node of one section and the first central node of an adjacent section. Cuff 260 extends between upper node 245 and inflow end 201. Thus, cuff 260 covers the cells of stent portion 207 formed by the struts between upper node 245 and inflow end 201, including diamond cell 228. FIG. 1J illustrates a side view of stent 200 including cuff 260 and an outline of leaflet 250b. In other words, the view of valve 206 in FIG. 1J is rotated about 60 degrees compared to the view of FIG. 1I. The view depicted in FIG. 1J is centered on vertical strut 210b showing approximately half of each of two adjacent stent sections 207a, 207b on each side of vertical strut 210b. Sections 207a, 207b surrounding vertical strut 210b are mirror images of each other. As described above, the cuff may be disposed on the stent's interior or luminal surface, its exterior or abluminal surface, and/or on both surfaces. A cuff ensures that blood does not just flow around the valve leaflets if the valve or valve assembly are not optimally seated in a valve annulus. A cuff, or a portion of a cuff disposed on the exterior of the stent, can help retard leakage around the outside of the valve (the latter known as paravalvular leakage or “PV” leakage). In the embodiment illustrated in FIGS. 1I-J, the cuff 260 only covers about half of the stent 200, leaving about half of the stent uncovered by the cuff. With this configuration, less cuff material is required compared to a cuff that covers more or all of the stent 200. Less cuff material may allow for the prosthetic heart valve 206 to crimp down to a smaller profile when collapsed. It is contemplated that the cuff may cover any amount of surface area of the cylinder formed by the stent. For example, the upper edge of the cuff may extend straight around the circumference of any cross section of the cylinder formed by the stent. Cuff 260 may be formed of any suitable material, including a biological material such as animal pericardium, or a synthetic material such as UHMWPE.

As noted above, FIGS. 1I-J illustrate a cuff 260 positioned on an interior of the stent 200. An example of an additional outer cuff 270 is illustrated in FIG. 1K. It should be understood that outer cuff 270 may take other shapes than that shown in FIG. 1K. The outer cuff 270 shown in FIG. 1K may be included without an inner cuff 260, but preferably is provided in addition to an inner cuff 260. The outer cuff 270 may be formed integrally with the inner cuff 260 and folded over (e.g., wrapped around) the inflow edge of the stent, or may be provided as a member that is separate from inner cuff 260. Outer cuff 270 may be formed of any of the materials described herein in connection with inner cuff 260. In the illustrated embodiment, outer cuff 270 includes an inflow edge 272 and an outflow edge 274. If the inner cuff 260 and outer cuff 270 are formed separately, the inflow edge 272 may be coupled to an inflow end of the stent 200 and/or an inflow edge of the inner cuff 260, for example via suturing, ultrasonic welding, or any other suitable attachment modality. The coupling between the inflow edge 272 of the outer cuff 270 and the stent 200 and/or inner cuff 260 preferably results in a seal between the inner cuff 260 and outer cuff 270 at the inflow end of the prosthetic heart valve so that any retrograde blood that flows into the space between the inner cuff 260 and outer cuff 270 is unable to pass beyond the inflow edges of the inner cuff 260 and outer cuff 270. The outflow edge 274 may be coupled at selected locations around the circumference of the stent 200 to struts of the stent 200 and/or to the inner cuff 260, for example via sutures. With this configuration, an opening may be formed between the inner cuff 260 and outer cuff 270 circumferentially between adjacent connection points, so that retrograde blood flow will tend to flow into the space between the inner cuff 260 and outer cuff 270 via the openings, without being able to continue passing beyond the inflow edges of the cuffs. As blood flows into the space between the inner cuff 260 and outer cuff 270, the outer cuff 270 may billow outwardly, creating even better sealing between the outer cuff 270 and the native valve annulus against which the outer cuff 270 presses. The outer cuff 270 may be provided as a continuous cylindrical member, or a strip that is wrapped around the outer circumference of the stent 200, with side edges, which may be parallel or non-parallel to a center longitudinal axis of the prosthetic heart valve, attached to each other so that the outer cuff 270 wraps around the entire circumference of the stent 200.

The stent may be formed from biocompatible materials, including metals and metal alloys such as cobalt chrome (or cobalt chromium) or stainless steel, although in some embodiments the stent may be formed of a shape memory material such as nitinol or the like. The stent is thus configured to collapse upon being crimped to a smaller diameter and/or expand upon being forced open, for example via a balloon within the stent expanding, and the stent will substantially maintain the shape to which it is modified when at rest. The stent may be crimped to collapse in a radial direction and lengthen (to some degree) in the axial direction, reducing its profile at any given cross-section. The stent may also be expanded in the radial direction and foreshortened (to some degree) in the axial direction.

The prosthetic heart valve may be delivered via any suitable transvascular route, for example including transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing the valve is inserted through the femoral artery and threaded against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstance, under an overlying sheath). Upon arrival at or adjacent the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

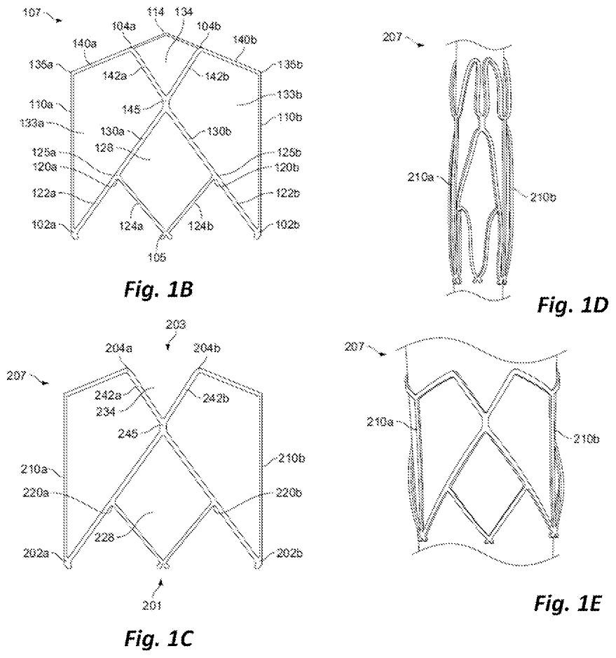

Referring to FIG. 2A, an example of a prosthetic heart valve PHV, which may include a stent similar to stents 100 or 200, is shown crimped over a balloon 380 of a balloon catheter 390 while the balloon 380 is in a deflated condition. It should be understood that other components of the delivery device, such as a handle used for steering and/or deployment, as well as a syringe for inflating the balloon 380, are omitted from FIGS. 2A-B. The prosthetic heart valve PHV may be delivered intravascularly, for example through the femoral artery, around the aortic arch, and into the native aortic valve annulus, while in the crimped condition shown in FIG. 2A. Once the desired position is obtained, fluid may be pushed through the balloon catheter 390 to inflate the balloon 380, as shown in FIG. 2B. FIG. 2B omits the prosthetic heart valve PHV, but it should be understood that, as the balloon 380 inflates, it forces the prosthetic heart valve PHV to expand into the native aortic valve annulus (although it should be understood that other heart valves may be replaced using the concepts described herein). In the illustrated example, fluid flows from a syringe (not shown) into the balloon 380 through a lumen within balloon catheter 390 and into one or more ports 385 located internal to the balloon 380. In the particular illustrated example of FIG. 2B, a first port 385 may be one or more apertures in a side wall of the balloon catheter 390, and a second port 385 may be the distal open end of the balloon catheter 390, which may terminate within the interior space of the balloon 380.

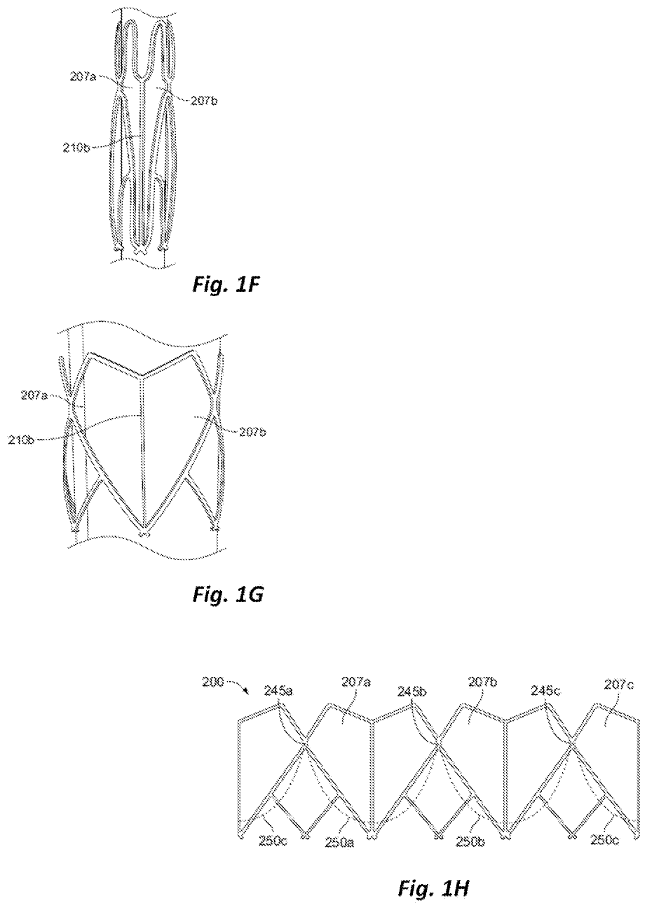

FIGS. 3A-C illustrate a prosthetic heart valve PHV having retention tabs that mate with a delivery device and/or a crimping tool. In FIGS. 3A-C the valve assembly is not prominently shown for the sake of clarity. Stent 200 of prosthetic heart valve PHV shows a vertical strut 210b defining approximately half of each of two adjacent stent sections 207a, 207b on each side of vertical strut 210b, with sections 207a, 207b surrounding vertical strut 210b being mirror images of each other. Stent 200 is similar to those previously discussed with reference to FIGS. 1A-K, and may include and of the features and/or material described above. In this example, retention tabs 310a,310b are disposed adjacent inflow end 201 and outflow end 203 of stent 200 stent, respectively. Specifically, circular retention tabs 310a are disposed at the end of the struts on outflow end 203, and circular retention tabs 310b are disposed at the end of the struts on inflow end 201. It will be understood that the retention tabs may be disposed adjacent inflow end 201 only, adjacent outflow end 203 only, or adjacent both the inflow and outflow ends. Additionally, it will be understood that though circular retention tabs are shown, the retention tabs may be square, rectangular, triangular or any other suitable shape. Additionally, the retention tabs may be in the form of hollow eyelets. In some examples, only a single retention tab is formed on the inflow end or the outflow end, or on both the inflow and outflow ends. Alternatively, each cell in an uppermost or lowermost row may terminate in a retention tab. In another variation, retention tabs are formed on every other cell in a row of cells (e.g., the retention tabs are formed on only half of the cells of either the inflow end, the outflow end or both).

In FIG. 3B, a delivery device 350 is shown with the prosthetic heart valve PHV loaded therein. In one exemplary embodiment, delivery device 350 includes an atraumatic tip 360, an inner shaft 356, a capsule 370 and a pair of hubs 354 having one or more receivers 355 for accepting the retention tabs of the prosthetic heart valve PHV. It will be understood that a one or two hubs 354 may be integrated into the delivery device at either the leading or trailing ends or both, and that the hubs 354 and/or receivers 355 may to correspond to the expected positions of the retention tabs on prosthetic heart valve PHV. Additionally, the number of receivers in each hub will correspond or equal to, the number of retention tabs on a corresponding prosthetic heart valve PHV. In some examples, the one or more receivers of the hub 354 includes a designated receiver 355 for each of the plurality of retentions tabs 310a,310b.

A similar arrangement may be applied to a crimping tool configured to evenly reduce the diameter of a prosthetic heart valve PHV as illustrated in FIG. 3C. Specifically, a crimping tool 395 may include one or more crimping hubs 364 that include one or more slots 365 to accept the one or more retention tabs 310 of the stent. By using one or more retention tabs 310 on the stent, and by utilizing receivers and/or slots on the delivery device and the crimping tool, respectively, it is possible to control the orientation by which the prosthetic heart valve PHV is loaded into both the delivery device and/or the crimping tool. Additionally, these techniques reduce variability and user error by clearly limiting the axial and/or radial movement of prosthetic heart valve PHV with respect to the delivery device, the crimping tool or both.

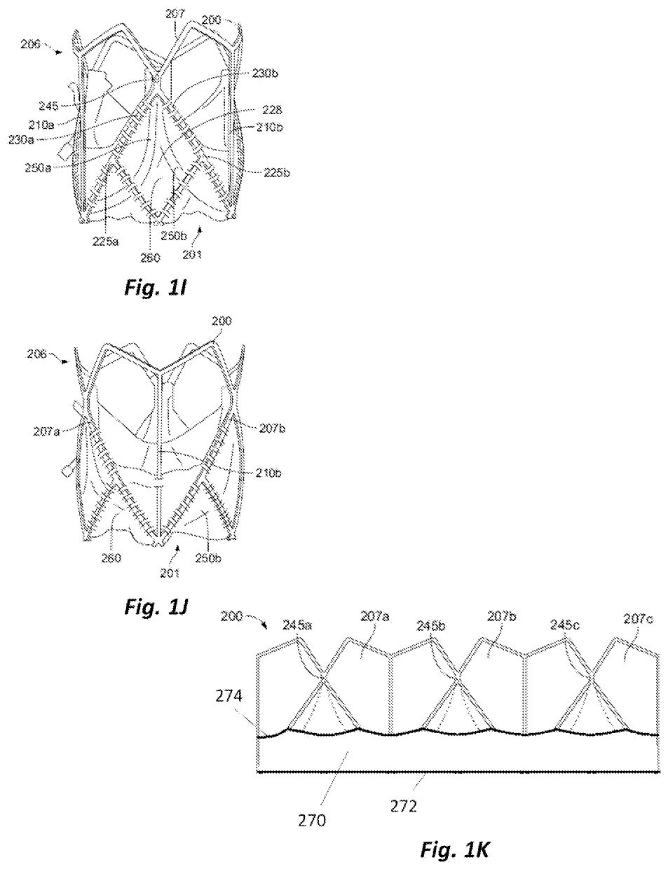

FIGS. 4A-B illustrate another example of prosthetic heart valve PHV in use with a valve cradle 475 that is used to secure and orient the valve during the crimping process. In this example, valve cradle 475 is in the form of a polymeric central support that is designed to sit within prosthetic heart valve PHV. In one example, valve cradle 475 is slightly shorter than prosthetic heart valve PHV, or is of equal height. As shown from above, valve cradle 475 may have a plurality of protrusions 478 alternating with a plurality of cavities 476, the plurality of cavities 476 being configured and arranged to receive portions of the plurality of leaflets during crimping. In the example, shown, three cavities 476 generally concave with smooth, rounded edges, and are arranged substantially 120 degrees apart to receive the bellies of the leaflets. The number of cavities 476 may be equal to the number of leaflets in a prosthetic heart valve PHV, or may be a multiple of the number of leaflets (e.g., two cavities for each leaflet, or three cavities for each leaflet). As prosthetic heart valve PHV is crimped and its diameter reduced, as shown in FIG. 4B, the leaflets (and in particular the belly sections of the leaflets) will gather or collect within the cavities so that consistent and predictable crimping of the valve assembly is achieved.

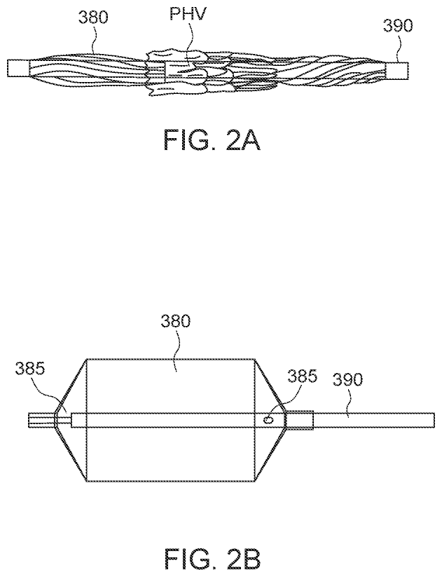

A garter system may also be used to orient, fix and/or stabilize prosthetic heart valve PHV with respect to a delivery device and/or a crimping tool. FIGS. 5A-7 are schematic representations of a prosthetic heart valve PHV in use with a garter system in connection with a delivery device. In FIG. 5A, a schematic representation of an expanded stent 500 is shown, the stent having hexagonal or diamond-shaped cells 530. In this example, a basic garter assembly 600 includes an enlarged anchor 610 connected to a coupler 605. Anchor 610 may be substantially circular or disk-shaped and may be sized to fit through cell 530 when the cell is substantially open (i.e., in the expanded condition of the stent). In one example, the diameter of the anchor 610 is smaller than one or more diagonals of an open cell, but larger than one or more diagonals of a partially or fully collapsed cell. Coupler 605 may include a band, wire, cord or suture, and may be attached to anchor 610 and may be affixed at its opposite end to a delivery device. In FIG. 5B, a schematic representation of a stent 500 is shown in its partially collapsed condition. In this state, anchor 610 is incapable of passing through closed cell 530 and the garter system will retain the stent as the anchor cannot pass through the closed or partially-closed cell. It will be understood that these techniques are equally applicable to a crimping tool.

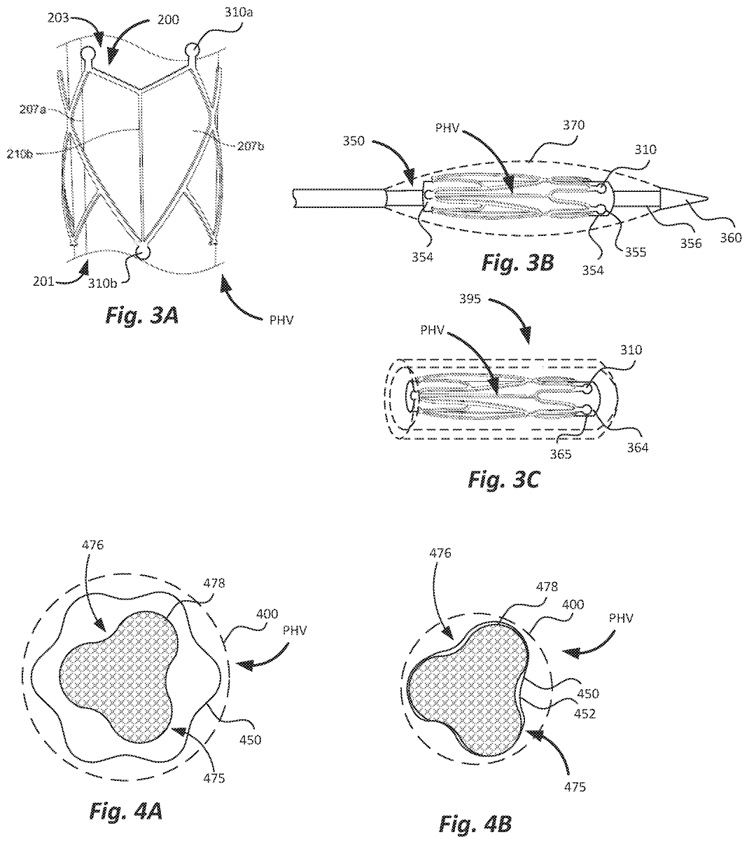

To more fully understand how the garter system works, FIG. 6A shows a prosthetic heart valve PHV seated on a delivery device 650 having an inner shaft 656 and balloon 670. For the sake of clarity, the valve assembly (e.g., the leaflets and cuff) are not shown. The system of FIG. 6A shows a garter system having four garter assemblies 600, two adjacent inflow end 501 and two adjacent outflow end 503 of prosthetic heart valve PHV. At inflow end 501, two garter assemblies lock the stent in place via anchors 610 that pass through cells. In the partially collapsed condition of the stent, shown in FIG. 6A, anchors 610 are incapable of passing through the collapsed or closed cells and are trapped thereby, and the couplers 605 join (e.g., adhere, affix or otherwise couple) to inner shaft 656, limiting the translational and/or radial movement of stent 500. Likewise, two garter assemblies fix the stent from outflow end 503. Any number of garter assemblies are possible including a single garter assembly, one garter assembly on each end of prosthetic heart valve PHV, or multiple garter assemblies on either or both ends, the garter assemblies being circumferentially arranged about the expected location of prosthetic heart valve PHV.

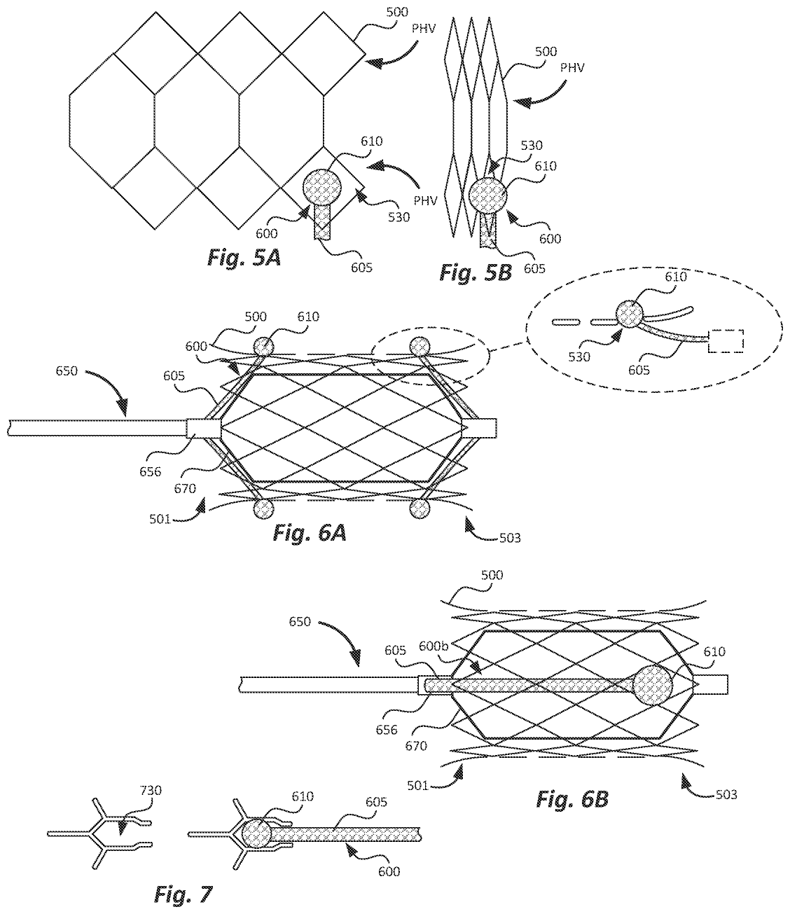

In another example, shown in FIG. 6B, a single garter assembly 600b is attached via a coupler 605 near inflow end 501 and the coupler extends toward outflow end 503 of stent 500, where anchor 610 will fasten to cell 530. As shown, coupler 605 extends along the axial length of prosthetic heart valve PHV, and is of a same or similar length as prosthetic heart valve PHV. It will be understood that more than one garter assembly similar to this may be disposed circumferentially about delivery device 650 so that the entire system includes one, two, three, four or more garter assemblies, circumferentially arranged. The opposite of this is also possible where a garter assembly is attached adjacent outflow end 503 and extends toward inflow end 501 along the length of prosthetic heart valve PHV. Additionally, instead of an anchor mating with a completely bounded cell, an unbounded cell 730 may be formed at the inflow and/or outflow ends of the stent to allow an anchor 610 to mate therewith and be more easily decoupleable during the delivery process (FIG. 7). As used herein, the term “unbounded” may mean any cell that is not completely circumscribed and that includes one more openings along its perimeter.

In another embodiment, a balloon may be textured, covered, or coated to provide a tacky or high-friction surface to prevent or limit movement of a prosthetic heart valve PHV thereon. In some examples, the coating may include a soft durometer material such as chronoprene, tecothane, polyurethane. Alternatively, a multi-layer balloon may be formed with a soft durometer on the outer diameter, such as 35D PEBAX® elastomer on the outer layer of a 74D PEBAX® elastomer base balloon. In at least some examples, the coating is applied to the balloon in the deflated state or a folded state so that the balloon is only partially coated with the tacky or high-friction substance.

In use, a prosthetic heart valve may be crimped, loaded and delivered according to any of the manners and configurations described above. First, a balloon-expandable prosthetic heart valve may be provided including a stent, a cuff and a plurality of leaflets, the cuff and the plurality of leaflets forming a valve assembly. The prosthetic heart valve PHV may be provided with retainers to aid in the placement, fixation and proper introduction of the prosthetic heart valve PHV within either the delivery device, a crimping tool, or both. A valve cradle may also be useful in gathering the leaflets appropriately and crimping prosthetic heart valve PHV in a consistent fashion. Instead of retainer, or in addition to them, a garter system may also be used to couple, orient, affix, or limit translation and/or rotation of a prosthetic heart valve PHV within a delivery device and/or crimping tool until the appropriate time for release. In some examples, the prosthetic heart valve PHV will naturally release itself from the garter system as the stent expands and the cells begin to open, which allows the anchors to pass therethrough and release the stent from the garter system.

According to one aspect of the disclosure, a prosthetic heart valve system, includes a prosthetic heart valve including a stent, a cuff and a plurality of leaflets, the cuff and the plurality of leaflets forming a valve assembly, and the stent having one or more retention tabs, and a delivery device having an inner shaft and an expandable balloon transitionable between a deflated state and an inflated state, the delivery device having a hub with one or more receivers to accept the one or more retention tabs.

According to another embodiment of the disclosure a prosthetic heart valve system, includes a prosthetic heart valve including a stent, a cuff and a plurality of leaflets, the cuff and the plurality of leaflets forming a valve assembly; and a delivery device having an inner shaft and an expandable balloon transitionable between a deflated state and an inflated state, the delivery device having a garter system including at least one anchor capable of mating with a cell of the stent, and at least one coupler connected to the at least one anchor.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A prosthetic heart valve system, comprising:
a prosthetic heart valve including a stent, a cuff and a plurality of leaflets, the cuff and the plurality of leaflets forming a valve assembly, and the stent having one or more retention tabs; and
a delivery device having an inner shaft and an expandable balloon transitionable between a deflated state and an inflated state, the delivery device having a hub with one or more receivers to accept the one or more retention tabs.

2. The prosthetic heart valve system of claim 1, wherein the stent includes a plurality of retention tabs disposed on an inflow end of the stent.

3. The prosthetic heart valve system of claim 1, wherein the stent includes a plurality of retention tabs disposed on an outflow end of the stent.

4. The prosthetic heart valve system of claim 1, wherein the one or more retention tabs includes a plurality of retention tabs, and the one or more receivers includes a designated receiver for each of the plurality of retentions tabs.

5. The prosthetic heart valve system of claim 1, further comprising a crimping tool having a crimping hub that includes one or more slots to accept the one or more retention tabs of the stent.

6. A prosthetic heart valve system, comprising:
a prosthetic heart valve including a stent, a cuff and a plurality of leaflets, the cuff and the plurality of leaflets forming a valve assembly, and the stent having one or more retention tabs;
a crimping tool sized to receive the prosthetic heart valve therein and to reduce a diameter of the stent; and
a valve cradle having a plurality of protrusions alternating with a plurality of cavities, the valve cradle being sized to fit within the stent, and the plurality of cavities being configured and arranged to receive portions of the plurality of leaflets during crimping.

7. The prosthetic heart valve system of claim 6, wherein the valve cradle includes three protrusions and three cavities.

8. The prosthetic heart valve system of claim 6, wherein each of the plurality of cavities of the valve cradle is sized and arranged to receive a respective belly of the plurality of leaflets.

9. The prosthetic heart valve system of claim 6, wherein the valve cradle is shorter than the prosthetic heart valve.

10. The prosthetic heart valve system of claim 6, wherein the valve cradle comprises a polymer.

11. A prosthetic heart valve system, comprising:
a prosthetic heart valve including a stent, a cuff and a plurality of leaflets, the cuff and the plurality of leaflets forming a valve assembly; and
a delivery device having an inner shaft and an expandable balloon transitionable between a deflated state and an inflated state, the delivery device having a garter system including at least one anchor capable of mating with a cell of the stent, and at least one coupler connected to the at least one anchor.

12. The prosthetic heart valve system of claim 11, wherein the at least one coupler joins the at least one anchor to the delivery device.

13. The prosthetic heart valve system of claim 11, wherein the at least one coupler joins the at least one anchor to the inner shaft of the delivery device.

14. The prosthetic heart valve system of claim 11, wherein the at least one anchor includes a plurality of anchors removably coupleable to an inflow end of the stent.

15. The prosthetic heart valve system of claim 11, wherein the at least one anchor includes a plurality of anchors removably coupleable to an outflow end of the stent.

16. The prosthetic heart valve system of claim 11, wherein the at least one anchor includes a plurality of anchors removably coupleable to both an inflow end and an outflow end of the stent.

17. The prosthetic heart valve system of claim 11, wherein each of the at least one anchor is substantially circular and sized to pass through an open cell of the stent when the stent is expanded, and to be trapped by a collapsed closed cell when the stent is at least partially collapsed.

18. The prosthetic heart valve system of claim 11, wherein the at least one coupler is coupled to the inner shaft of the delivery device adjacent an inflow end of the stent and extends to an outflow end of the stent.

19. The prosthetic heart valve system of claim 11, wherein the at least one coupler is coupled to the inner shaft of the delivery device adjacent an outflow end of the stent and extends to an inflow end of the stent.

20. The prosthetic heart valve system of claim 11, wherein the at least one coupler comprises multiple couplers circumferentially arranged about the stent.
